(19) United States
(12) Patent Application Publication
(10) Pub. No.: US 2003/0083301 A1
(43) Pub. Date: May 1, 2003

(54) Therapeutic treatments for spinal cord injury via blockade of interleukin-1 receptor

(75) Inventors: Regino Perez-Polo, Galveston, TX (US); Olivera Nesic, Galveston, TX (US)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701-3271, US

(73) Assignee: Board of Regents, The University of Texas System

(21) Appl. No.: 10/236,869

(22) Filed: Sep. 6, 2002

Related U.S. Application Data
(60) Provisional application No. 60/317,778, filed on Sep. 6, 2001.

(51) Int. Cl. A61K 48/00; C12N 15/85; A61K 38/17

(52) U.S. Cl. 514/44; 514/12; 435/455

(57) ABSTRACT

The present invention concerns the use of interleukin-1 receptor antagonists for the treatment of spinal cord injuries (SCI). IL-1ra may be administered to a patient with a spinal cord injury in an effective amount. Such methods also include combination therapies in which IL-1ra is administered in addition to other therapeutic agents for the treatment of SCI.

This applications claims priority to U.S. Provisional Patent Application No. 60/317,778, filed on Sep. 6, 2001, which is specifically incorporated by reference herein. The government may own rights in the present invention pursuant to grant number 1 PO1 NS39161 from the National Institute of Neurological Disorders and Stroke.

### BACKGROUND OF THE INVENTION

1. Field of the Invention

The present invention relates generally to the fields of neurology and physiology. More particularly, it concerns treatment methods for patients with spinal cord injuries in which motor recovery is possible, involving blocking IL-1β, such as with IL-1 receptor antagonist.

2. Description of Related Art

In the United States, there are approximately 450,000 people who have endured a spinal cord injury (SCI). There are approximately 10,000 new cases each year. Many of these SCI sufferers involve males between the ages of 16-30 (82%). Of these, motor vehicle accidents (36%), violence (28.9%), or falls (21.2%) account for a large number. There is currently no cure for SCI.

Spinal cord injury (SCI) causes tissue disruption, cellular energy deficit, and glutamate surges followed by a progressive neurodegeneration that is characterized by the loss of neurons and glia due to delayed cell death, via both apoptosis and necrosis.

A critical component of neurodegeneration after SCI is an inflammatory response in which cytokines play a significant role. For example, levels of interleukin-1 (IL-1α and IL-1β), a major pro-inflammatory cytokine, increase rapidly in the spinal cord after traumatic injury (Wang et al., 1997; Klusman and Schwab, 1997; Streit et al., 1998; Hayashi et al., 2000). IL-1β seems to be the main IL-1 isoform induced in the CNS in response to injury (Rothwell et al., 1997, Rothwell, 1999). IL-1β mRNA levels increase within 6 h after injury and then decrease after 24 h (Wang et al., 1997; Klusman and Schwab, 1997; Hayashi et al., 2000). It is likely that the early increase in IL-1β mRNA levels originates from spinal cord microglial cells (Bartholdi and Schwab, 1997). What triggers this rapid transient increase in the IL-1β mRNA levels in the contused spinal cord is not known. It is known, however, that a robust increase in cytokines does not occur after CNS injuries if the blood-brain barrier remains intact (Streit et al., 1998). One explanation for this phenomenon is that serological factors that are prevented from entering the uninjured CNS could serve as signals that stimulate transcription of IL-1β after trauma (Hetier et al., 1988). Also, since injury to the spinal cord releases glutamate; thus, increasing extracellular glutamate to high levels (Liu et al., 1997) and glutamate induces IL-1 synthesis (Vezzani et al., 1999), it is not surprising that SCI stimulates IL-1 mediated inflammatory cascades. Despite the presence of stimulatory signals for increased IL-1 mRNA transcription by variety of agents most of the IL-1β mRNA is degraded relatively quickly because of mRNA instability (Dinarello, 1998a) and/or the presence of transcriptional repressors (Lebedeva and Singh, 1997). Following synthesis, pro-IL-1β remains primarily in the cytosol until cleaved and transported out of the cell. The release of mature Il-1β depends on the cleavage of aspartic-alanine peptide bonds by IL-1β converting enzyme (ICE). ICE also cleaves and activates Il-18, another member of IL-1 family, but, not IL-1α (Dinarello, 1999). However, Caspase-1 is not the only enzyme that can cleave proIL-1β, granzyme A does that as well (Irmler et al 1995). Thus, the production of IL-1β is carefully regulated, not only by ICE activity, but also via several mechanisms, as evidenced by the complex promoter region of the IL-1β gene (Fenton, 1992). Although, spinal cord injury in rats does not affect the baseline activity of Caspase-1 (Springer et al., 1999), the levels of Il-1β increase significantly (Wang, 1997). Levels of IL-1β protein in the spinal cord at the lesion site increase 1 hour after injury, peak at 8 hours and remain elevated for up to 7 days after injury (Wang et al., 1997).

IL-1β is persistently elevated in chronic inflammatory conditions that can result in neurodegeneration (Griffin et al., 1989). Furthermore, there is evidence that IL-1β mediates neurodegeneration in Down's syndrome, Alzheimer's disease, Parkinson's disease, multiple sclerosis and scrapies (see Rothwell, 1999). The most direct evidence implicating endogenous IL-1 in neurodegeneration derives from experiments that employed a recombinant IL-1 receptor antagonist (IL-1ra). IL-1ra is a selective endogenous receptor antagonist that blocks the actions of IL-1α and IL-1β (Dinarello, 1998b; Arend et al., 1999). Injection or overexpression of IL-1ra significantly inhibits neuronal damage induced in rodents by focal cerebral ischemia or excitotoxin administration (see Rothwell, 1999). Moreover, the administration of exogenous IL-1β markedly exacerbates ischemic or excitotoxic brain injury (Loddick and Rothwell, 1996; Lawrence et al., 1998). Although the precise mechanism by which IL-1β achieves this effect is unknown, it may be via IL-1β stimulation of apoptosis. There are a small number of observations that directly connect IL-1 expression to apoptotic cell death (Friedlander, 1996, 1997). It has also been shown that the inhibition of caspase-1 and the subsequent decrease in the production of IL-1 reduces the apoptosis induced by cerebral ischemia (Rabuffetti et al., 2000). Recently, Holmin and Mathiesen (2000) found that intracerebral administration of IL-1β induces apoptosis, as detected by the TUNEL technique. They also found that TUNEL positive cells expressed higher levels of the pro-apoptotic Bax gene than the anti-apoptotic Bcl-2 gene.

The appearance of apoptosis after SCI has been well documented (Liu et al., 1997; Crowe et al., 1997; Emery et al, 1998). However, the mechanisms responsible for apoptosis after SCI have only recently been investigated in detail. Springer et al. (1999) have shown that SCI activates caspase-3, which in turn cleaves several essential downstream substrates involved in the expression of apoptotic phenotypes. They found that caspase-3 is first activated in neurons and at later times in oligodendroglia. Furthermore, Saito et al. (2000) demonstrated that there is an upregulation of the key apoptotic mediators p53 and Bax in oligodendrocytes, microglia and astrocytes, but not in spinal cord neurons spinal after injury. Apoptosis occurs in spinothalamic tract (STT) neurons following SCI and the detected apoptosis correlates well with the observed downregulation of bcl-x gene expression (Qiu et al., submitted for publication). Thalamic deafferentation by the loss of STT cells may account for the development of chronic pain in the majority of SCI patients (Tasker and Dostrovsky, 1989; Hulsebosch et al., 2000).

The development of innovative therapeutic strategies that ameliorate the damage caused when a patient is subjected to a spinal cord injury is needed.

### SUMMARY OF THE INVENTION

The present invention concerns interfering with the interleukin 1 receptor (IL-1R) as a way of effecting treatment for spinal cord injury. It takes advantage of the observation that IL-1β expression is increased in cells of the spinal cord soon after there is an injury to the spinal cord. The present invention concerns treatment of SCI that targets both neurons and glial cells, which includes oligodendrocytes, astrocytes, and microglial cells (collectively referred to as “neuronal cells”). Furthermore, because there is a motor recovery benefit from the methods of the invention, in some embodiments, treatment is directed to SCI in which the spinal cord is not completely severed or transected.

Methods of treating spinal cord injury include administering a therapeutically effective amount of IL-1 receptor antagonist (IL-1ra) to the site of injury. Subjects who may be treated include any organism with a spinal cord, including any mammal, such as a human, horse, dog, cow, cat, or rat. In some embodiments of the invention, the step of first identifying a subject in need of such treatment is part of the method. Identifying such a subject may include evaluating or assaying for neurologic symptoms or employing other physiological tests to determine whether a subject has SCI. If a person is determined to have SCI in which the spinal cord is not fully severed (cut), then treatment methods of the invention may be implemented with respect to that subject. The term “therapeutically effective” refers to a specific parameter, such as amount or time, that achieves a therapeutic benefit with respect to SCI. A therapeutic benefit means the subject experiences a ramification of the treatment that may include, but is not limited to, any reduction in pain, recovery of motor function, increase in ability to feel, faster recovery, amenability to other treatment or rehabilitation, inhibition of neurodegeneration, inhibition of apoptosis of neuronal cells, decrease in neurologic symptoms, prevention of neurologic symptoms, increased life span, reduction in risk of conditions associated with SCI—such as pneumonia, bed sores, and other known ailments, or reduction in amount or extent of other SCI therapy.

The invention concerns blocking IL-1 receptor-mediated transduction. Thus, included in some embodiments of the invention are inhibitors of IL-1 receptor, including IL-1ra. In some embodiments, the invention concerns other general and specific antagonists of IL-1 receptor, including small chemical molecules and proteinaceous molecules. Antagonists may further include nucleic acid molecules that encode antagonists (proteinaceous molecules) or inhibit IL-1 receptor themselves, such as ribozymes or antisense molecules complementary or identical to IL-1 receptor-encoding nucleic acids. In some embodiments of the invention, the inhibitor of IL-1 receptor is IL-1ra, also known as IRAP. The terms are used interchangeably. One may also use the methods described herein, particularly the examples to provide the basis for a screening method to identify inhibitors of IL-1 receptor. A functional assay from the examples is to evaluate for apoptosis of neuronal cells in the presence of a candidate compound. Thus, compounds with inhibitory activity against IL-1 receptor may be used in methods of the invention as IL-1ra is used.

In some embodiments of the invention, IL-1ra is purified from cells in which it is expressed from a chromosomal IL-1ra gene (encoded by genomic DNA). Alternatively, IL-1ra may be recombinant, that is, produced through the use of recombinant DNA technology. Various mammalian version of recombinant IL-1ra are contemplated as part of the invention, including recombinant mouse and recombinant human IL-1ra. Recombinant IL-1ra or natural IL-1ra may be purified from cells or tissue and administered to the subject. Alternatively, a subject may be given a nucleic acid comprising a nucleic acid sequence that encodes all or part of recombinant IL-1ra. In some embodiments, the nucleic acid is a vector. In some embodiments the vector is a plasmid. In other embodiments the vector is a viral vector. Viral vectors may be derived from adenovirus, lentivirus, adeno-associated virus, retrovirus, herpesvirus, or vaccinia virus. In some methods an adenovirus vector is specifically employed to deliver an inhibitor of IL-1 receptor, including IL-1ra.

In still further embodiments of the invention, methods include identifying the type of spinal cord injury. This may include visual examination with or without optical aids or other types of tests that allow the nature of injury to be assessed. Such tests and examinations are well known to those of ordinary skill in the art. Furthermore, tests that evaluate or assay neurologic symptoms or other neurological damage may be implemented to identify the type of spinal cord injury. It is presumed that following prognosis and treatment steps, patients may be monitored for physiological response, as well as toxicity and overall health. Patients may also undergo additional therapy, including conventional rehabilitation through physical therapy.

The spinal cord injury may be traction or contusion on the spinal cord or it may be a partial transection of the spinal cord. Other types of trauma to the spinal cord are included, particularly if they involve damage to both neurons and glial cells.

In additional embodiments of the invention, methods of treatment include administration of the inhibitor of IL-1 receptor over a certain time period, such as a therapeutically effective time period. In some embodiments, the administration of IL-1ra or a nucleic acid encoding IL-1ra is specifically contemplated. It is contemplated that the time period, in some embodiments, is within one hour of the time of the spinal cord injury to 72 hours after the spinal cord injury. In additional embodiments, the time period begins within about 1 hour of injury to 72 hours after the spinal cord injury. In still further embodiments, the time period is between 24 hours and 72 hours in length or is between 3 and 6 days in length. It is contemplated that the treatment may be administered within or after 1, 2, 3, 4, 5, 6, 7, 8, 9, 10, 11, 12, 13, 14, 15, 16, 17, 18, 19, 20, 21, 22, 23, 24 hours or 1 2, 3, 4, 5, 6, 7 days or 1, 2, 3, 4, 5 weeks, or 1, 2, 3, 4, 5, 6, 7, 8, 9 10, 11, 12 months the occurrence of the injury. Also, it is contemplated that the treatment may be administered for 1, 2, 3, 4, 5, 6, 7, 8, 9, 10, 11, 12, 13, 14, 15, 16, 17, 18, 19, 20, 21, 22, 23, 24 hours or 1 2, 3, 4, 5, 6, 7 days or 1, 2, 3, 4, 5 weeks, or 1, 2, 3, 4, 5, 6, 7, 8, 9 10, 11, 12 months or more. Chronic administration is contemplated for 5, 10, 15, 20, 25, 30, 35, 40, 45, 50, 55, minutes or 1, 2, 3, 4, 5, 6, 7, 8, 9, 10, 11, 12, 13, 14, 15, 16, 17, 18, 19, 20, 21, 22, 23, 24 or more hours. Multiple administrations are also contemplated. In some embodiments the administration is given at least twice (repeated at least once).

A subject may be administered different amounts of therapeutic agents. In some embodiments, the amount of an IL-1 receptor antagonist that is administered is between 1 and 1000 nanograms per kilogram body weight per hour. In other embodiments, the amount is between 1 and 100 nanograms per kilogram body weight per hour or between 1 and 10 nanograms per kilogram body weight per hour. It is contemplated that dosages maybe 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, 5000 nanograms per kilogram body weight per hour (ng/kg/hr). It is also contemplated that dosages may also be at least and/or not more than those same amounts.

In some embodiments the inhibitor of the IL-1 receptor is dispersed or dissolved in a pharmaceutically acceptable carrier. IL-1ra is specifically contemplated as dispersed or dissolved in a pharmaceutically acceptable carrier.

Inhibitors of IL-1 receptor may be administered to a spinal cord injury. In some embodiments, an IL-1 receptor antagonist is administered to the site of injury via a catheter. It is contemplated that the pump may be connected to the catheter. Administration may also be by perfusion, direct injection, or lavage.

As discussed above, other therapies may be implemented with IL-1 receptor inhibitors, such as IL-1ra. Some embodiments also include administering methylpredisolone, pain relievers or pain killers, or neurotrophic factors, any of which may be administered before, after, or simultaneously with the IL-1ra. Pain management may be implemented with any methods of the invention. Neurotrophic factor that may be administered or that are comprised in compositions of the invention include, but are not limited to, is bFGF, aFGF, CNTF, NGF, BDNF, GDNF, NT3, NT4/5, IGF-1, IGF-II, NT-4, IL-1β, TNFα., TGF-β, TGF-β1, NTN, persephin, artemin, or AL-1. It is contemplated that one or more of the factors may be administered to a subject during the course of therapy with IL-1ra. The inclusion of NT3 is specifically contemplated in embodiments of the invention. Par4, a pro-apoptotic molecule, may also be inhibited in some embodiments of the invention. This may or may not be implemented in conjunction with NT3 therapy. In some embodiments, Par4 is inhibited by a Par4 antisense molecule.

Thus, in further embodiments, there are pharmaceutical compositions comprising IL-1 receptor antagonist and methylpredisolone. Other compositions include IL-1 receptor antagonist and a neurotrophic factor, such as those discussed in the previous paragraph, and/or a Par4 inhibitor. A Par4 inhibitor is an antisense molecule in some embodiments of the invention. Such compositions may be administered to patients in need of such therapy, include patients with SCI.

It is contemplated that any aspect of the invention discussed in the context of one embodiment may be applied with respect to any other embodiment of the invention, and vice versa.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Abundance of IL-1β mRNA among injured (hatched bars) and uninjured spinal cords (solid bars). IL-1β mRNA was measured 1 h, 6 h, 24 h and 72 h after injury. The expression levels were determined by using mRNA Ribonuclease Protection Assay arrays. All levels of IL-1β mRNA values are relative to the expression of the housekeeping gene L32. The injury was performed at spinal segment T8 and the levels of IL-1β mRNA were measured at spinal segments caudal (T7, T6, T5) from the site of injury and rostral (T9, T10, T11) (n=3; the presented values are from one measurement).

FIG. 2. Time course measurements of IL-1β protein and mRNA concentrations. IL-1β mRNA was measured 1 h, 6 h, 24 h and 72 h after injury. Rat spinal cord was removed for analysis after contusion injury (n=3) or sham treatment (n=5). IL-1β protein concentration was measured with an IL-1β ELISA and expressed as pg/ml of IL-1β per μg of total protein. The expression levels were determined by using mRNA Ribonuclease Protection Assay arrays. All levels of IL-1β mRNA values are relative to the expression of the housekeeping gene L32.

FIG. 3. Quantitative evaluation of apoptosis after SCI assessed by monitoring histone associated DNA fragments in spinal cord cytoplasmic fractions. The cytosolic fractions of spinal cord segments T5 and T8 (site of injury) were isolated from sham animals and from contusion injured animals sacrificed after 72 h. The first column represents levels of apoptosis in tissues from sham injury animals (n=6). The second and third columns represent the levels of apoptosis in the injured rats (n=6) without treatment (second column) or chronically treated with rmIL-1ra (750 ng/ml) for 72 h (third column).

FIG. 4. Caspase-3 activity was determined by measuring the cleavage of the substrate consisting of the tetrapeptide (DEVD) and a chromogen in the spinal cord homogenates 72 h after injury in rats injected with vehicle (first two columns) or with rmIL-1ra (750 ng/ml; third column) for 72 h.

FIG. 5. The locomotor recovery scores (averaged across hindlimbs) of sham-treated rats (n=7, filled circles) and rats treated with 750 ng/ml rmIL-1ra for 72 h (n=6, triangles) over a period of 30 days. Scored on the BBB scale in an open field test. A score of 21 points reflects normal locomotion.

FIG. 6. L-6 mRNA levels measured by RPA in six injured and three sham rats, at two time points (1 and 6 hours after injury).

FIG. 7. Schematic representation of gene expression changes in sham (first column in the corresponding graph), injured (middle column) and IL-1ra treated injured spinal cords (third column) detected for some of the ROS-induced genes. Most likely, IL-1-induced iNOS and COX-2 transcriptional changes are mediated via IL-1 receptor activation of NF-kB (Xie et al., 1994; Nakao et al., 2000). ROS typically induce transcription of genes with protective antioxidant activity: Hypoxia Inducuble Factor (HIF) (Semenza, 2000), Heme Oxygenase (Lee et al., 1999) and Manganese-Containing Superoxide Dismustase (MnSOD; Franco et al., 1999).

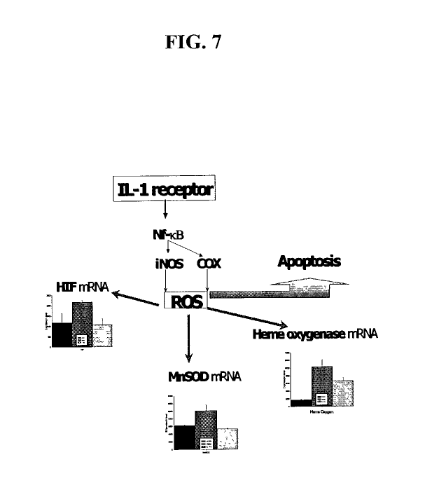

### DESCRIPTION OF ILLUSTRATIVE EMBODIMENTS

I. Spinal Cord Injury

Contusion impact to the spinal cord produces a primary lesion at the site of trauma and a delayed secondary lesion extending rostro-caudally from the injured site. This trauma-induced neurodegeneration leads to the progressive loss of motor and sensory functions (Basso et al, 1996; Taoka and Okajima, 1998). The absence of an efficient therapy for patients suffering SCI reflects, in part, a lack of understanding of the neurodegenerative events induced by SCI.

Neurodegeneration in the CNS results in outcomes that include variant forms of cell death whose properties are typically ascribed to either necrosis or apoptosis (Martin et al., 1998). The potential contribution of the inflammatory response to enhanced CNS cell death has not been fully explored, although inflammation has been well documented in a number of neurodegenerative conditions like Alzheimer's disease, AIDS related dementia (McGeer and Rogers, 1992, Gendelman et al., 1994, respectively) and acute CNS traumas (Clark et al., 1994; Dusart and Schwab, 1994). Furthermore, the only currently available treatment for SCI is the anti-inflammatory drug methylpredisolone, consistent with the presence of an inflammatory component in progressive neurodegeneration after SCI (Short et al, 2000).

SCI-induced inflammation begins when cytokines and antigenically active denatured intracellular debris, released from injured spinal cord cells, activate resident microglia. Activated microglia synthesize and release the pro-inflammatory cytokines that attract neutrophils, which in turn release toxic reactive oxygen species (ROS) and degradative enzymes. Thus, the accumulation of destructive neutrophils exacerbates lesion after SCI (Clark et al., 1994; Dusart and Schwab, 1994). However, it is not known whether the inflammatory cascade (optimal in the protection of CNS against infectious pathogens) triggered by SCI is beneficial or detrimental (Raivich et al., 1999). Although, all stages of inflammation are regulated by cytokines, their contribution to the recovery, or exacerbation of CNS injury remains to be established. Thus, the progressive neurodegeneration that follows CNS trauma may result from an overproduction of proinflammatory cytokines.

Injury to the spinal cord triggers a rapid and robust upregulation of proinflammatory cytokines that includes IL-1β (Wang et al 1997; Klusman and Schwab, 1997; Hayashi et al., 2000). Wang et al. (1997) have shown that IL-1β protein levels also increase by day one after spinal injury and stay elevated for 7 days after injury. However, in our study, IL-1β protein levels increased significantly as early as 6 h after injury, and then decreased by the third day after injury. Tonai et al. (1999) have also found that IL-1β protein levels peak within 4 h after spinal injury, and decline by 24 h.

Apoptosis of neurons and glia has been shown to occur after SCI (Katoh et al., 1996; Crowe et al., 1997; Liu et al., 1997; Wada et al., 1999; Nakahara et al., 1999). Apoptosis does not occur at 6 h after injury, but does occur 24 h after injury (Qiu et al., submitted for publication), consistent with published reports (Yong et al., 1998; Wada et al., 1999; Springer et al., 1999).

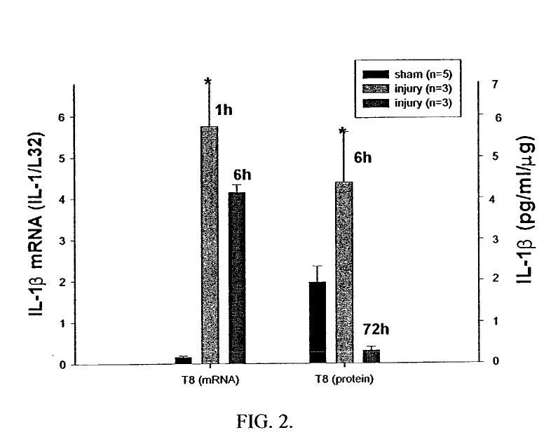

More direct evidence suggesting a causal connection between IL-1 and apoptosis was obtained from the observations of the effect of IL-1ra. It has been documented that recombinant IL-1ra (rIL-1ra) treatment protects against ischemic and hypoxic CNS injuries (Martin et al., 1994; Loddick and Rothwell, 1996; Relton et al., 1996). For example, Sanderson et al. (1996) have shown that the systemic administration of rhIL-1ra attenuates neuronal loss after a fluid-percussion brain injury. However, the effect of rIL-1ra on cell death in SCI had not been tested. The chronic administration of IL-1ra significantly reduced cell death after SCI (FIG. 2A), resulting in there being no significant differences in the levels of cell death observed in sham-treated vs. injured spinal cords injected with IL-1ra.

The execution of apoptosis is most often mediated through the activation of caspases (Li and Yuan, 2000). Moreover, Springer et al. (1999) have shown that caspase-3 and upstream and downstream components of the caspase-3 pathway are activated after traumatic injury to rat spinal cord. IL-1 has not been previously connected with caspase-3 activity in spinal cord injury.

Basso et al. (1996) have shown that sparing as few as 5-10% of the fibers at the lesion center is sufficient to preserve locomotor function after SCI. The excess IL-1β levels around the site of spinal cord injury trigger apoptosis, which then limits locomotor recovery. The present invention's methods of treatment to inhibit IL-1β in the variety of cells that are involved in spinal cord injury contribute to improving locomotor recovery. Thus, the term “spinal cord injury” as used herein refers to a spine that has been injured in some way, but does not include spinal cords that have been completely severed or transected, because such spinal cords are not contemplated to benefit from the therapy of the invention, which effects an improvement in motor function.

SCI is understood as damage to the spinal cord that results in a loss of function, for example mobility or the physical ability to feel. SCI may also result in neurologic symptoms, which include: amaurosis fugax, aphasia, coma, confusion, dementia, delirium, diplopia, dysphagia, difficult speech, hyperesthesia, lack of coordination, lethargy, localized weakness, numbness, paralysis, seizures, tingling, tremor, and walking difficulties. Typical causes of damage are trauma (falls, car accidents, etc.) or disease (polio, spina bifida, Friedreich's Ataxia, etc.). With most people with SCI, the spinal cord is intact (greater than 90%), but the damage to it results in loss of functioning. Contusions to the spinal cord also qualify as SCI. Ischemia may produce some of the same neurologic symptoms of SCI, but it results from a decrease in the blood supply to an organ, particularly the brain. For purposes of the invention, spinal cord injury does not include, in most embodiments, ischemia.

A. Current Treatments

Methylprednisolone is a corticosteroid, commercially available as Medrol®, Meprolone®. It helps to reduce swelling, redness, itching, and allergic reactions. In addition to being used for injury to the spinal cord, it can be used to treat severe allergies, skin problems, asthma, arthritis, multiple sclerosis and other conditions.

B. IL-1 Receptor Antagonists

A wide range of IL-1 receptor antagonists are available for use in the present invention. The term “IL-1 receptor antagonist” refers to a proteinaceous molecule that specifically binds to IL-1 receptor, either IL-1α or IL-1β, but does not transduce a biological response or signal. Among them are those derived from natural sources or produced as products of recombinant technology. Included in these are complete IL-1 receptor antagonist proteins, homologs and analogs thereof, biologically active fragments of complete proteins, and precursors to biologically active IL-1 receptor antagonists. By way of example, these include, but are not limited to those derived from humans, mice, rats, rabbits, dogs, horses, cows, and bottlenose dolphins. These exemplary proteins and nucleotide sequences encoding them are publically available through the National Center for Biotechnology Information (http://www.ncbi.nlm.nih.gov), indexed by accession numbers as follows: for humans, NP000568, P18510, P27930, P14778, NP036407, AAC13499, AAB92270, AAB92269, AAB92268, I37893, CAA59087, AF186094, AJ005835, AF057168, U65590, X77090, X52015; for mice, NP112444, A44610, P27931, P13504, P25085, AAC13499, AAC15251, AAA39309, NM031167, AJ250429, BB144147, S64082, M64404; for rats, P25086; for dogs, AF216526, AY026462, AAG36777, AY026462; for horses, AAD51441, AF186094, AF088186, AF072476, O18999; for cows, BAA31854, O77482; for bottlenose dolphin, BAB11806, rat, P25086, rabbit, P26890, and pig, Q29056, the contents and disclosure of which are all specifically incorporated herein by reference. Other IL-1ra sequences could found using search techniques and databases well known to those of skill in the art.

Other IL-1 receptor antagonists and methods contemplated for use as part of the invention include those described in the following, which are specifically incorporated by reference: U.S. Pat. Nos. 6,268,142; 6,168,791; 6,159,460; 6,090,775; 6,063,600; 6,036,978; 6,054,559; 5,922,573; 5,863,769; 5,858,355; 5,863,769; 5,508,262; and 6,013,253.

IL-1 receptor antagonist (IL-1ra), also known as IL-1 receptor antagonist protein (IRAP) is a natural antagonist of IL-1α and IL-1β. It binds to the IL-1 receptor but does not transduce an intracellular signal or a biological response. At least three alternatively splice forms of IL-1ra exist: one encodes a secreted protein, and the other two encode intracellular proteins.

C. Neurotrophic Factors

“Neurotrophic factors” are growth factors that promote differentiation, maintain a mature phenotype and provide trophic support, promoting growth and survival of neurons. Neurotrophic factors reside in the nervous system or in innervated tissues. The following have been described as neurotrophic factors: basic fibroblast growth factor, (bFGF), acidic fibroblast growth factors (aFGF), ciliary neurotrophic factor (CNTF), nerve growth factor (NGF), brain derived neurotrophic factor (BDNF), glial-derived neurotrophic factor (GDNF), neurotrophin-3 (NT3), NT4/5, insulin-like growth factor (IGF-1), IGF-II, NT-4, IL-1.β, TNFα., transforming growth factor β (TGF-β, TGF-β1), neurturin (NTN), persephin (PSP), artemin, and AL-1. Uses of neurotrophic factors are well known to those of skill in the art and can be found, for example, in U.S. Patent Publication No. 20010011126, U.S. Pat. Nos. 6,284,540, 6,280,732, 6,274,624, 6,221,676, which are incorporated by reference herein. The use of Par4 is well known to those of skill in the art, as disclosed in U.S. Pat. No. 6,111,075, which is hereby incorporated by reference.

It is contemplated that a neurotrophic factor or factors may be administered as part of a therapy with an IL-1ra therapy for treatment of spinal cord injury.

II. Proteinaceous Compositions

In certain embodiments, the present invention concerns compositions comprising a proteinaceous molecule that blocks IL-1β, such as an IL-1 receptor antagonist. Other embodiments of the invention concern the administration of NT3, a neurotrophic factor, which may be administered as a protein or as an NT3-encoding nucleic acid.

As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. It will be understood that any reference to one type of proteinaceous molecule, such as “polypeptide,” refers to the other types as well. Thus, discussion about methods of “protein purification” apply equally to purification of any proteinaceous molecule, such as a peptide. All the “proteinaceous” terms described above may be used interchangeably herein. Furthermore, these terms may be applied to fusion proteins or protein conjugates as well.

In certain embodiments the size of at least one proteinaceous molecule may comprise, be at most, or be at least (but is not limited to) 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 350, 400, 450, 500, 550, 600, 650, 700, 750, 800, 850, 900, 950, 1000, 1100, 1200, 1300, 1400, 1500, 1750, 2000, 2250, 2500 or greater amino molecule residues, and any range derivable therein. Such sizes are applicable with respect to a proteinaceous molecule that includes all or part of an IRAC peptide or polypeptide, or all or part of IL-IL1, which has 47% identity with IRAC.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

The present invention also describes IL-1ra peptides. The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Peptides with at least about 10, 11, 12, 13, 14, 15, 16, 17, 18, 19,20,21, 22,23, 24, 25, 26, 27,28, 29, 30, 31, 32, 33, 34, 35, 36, 37, 38, 39, 40, 41, 42, 43, 44, 45, 50, 55, 60, 65, 70, 75, 80, 85, 90, 95 or up to about 100 amino acid residues are contemplated by the present invention.

The compositions of the invention may include a peptide comprising a IL-1ra that has been modified to enhance its activity or to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides that include all L-amino acids, all D-amino acids, or a mixture thereof. The use of D-amino acids may confer additional resistance to proteases naturally found within the human body and are less immunogenic and can therefore be expected to have longer biological half lives.

1. Functional Aspects

The present invention concerns peptides and polypeptides that inhibit IL-1 receptors such as IL-1ra. Thus, when the present application refers to the function or activity of “IL-1ra” one of ordinary skill in the art would understand that this includes polypeptides and proteins that can specifically compete with IL-1α and IL-1β for binding to IL-1 receptor. Determination of activity may be achieved using assays familiar to those of skill in the art, particularly with respect to the polypeptide's or peptide's activity, and may include for comparison purposes, for example, the use of native and/or recombinant versions of it.

2. Modified Proteins

Modified proteins of the present invention may possess deletions and/or substitutions of amino acids; thus, a protein with a deletion, a protein with a substitution, and a protein with a deletion and a substitution are modified proteins. In some embodiments these modified proteins may further include insertions or added amino acids, such as with fusion proteins or proteins with linkers, for example.

Substitutional or replacement variants typically contain the exchange of one amino acid for another at one or more sites within the protein and may be designed to modulate one or more properties of the polypeptide, particularly to reduce its immunogenicity/antigenicity, reduce any side effects in a subject, or increase its efficacy. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. An antigenic region of a polypeptide may be substituted for a less antigenic region; the less antigenic region may contain residues that are identical to the corresponding residues in the native protein, yet also contain some conservative substitutions and/or nonconservative substitutions.

In addition to a deletion or substitution, a modified protein may possess an insertion of residues, which typically involves the addition of at least one residue in the polypeptide. This may include the insertion of a targeting peptide or polypeptide or simply a single residue. Terminal additions, called fusion proteins, are discussed below.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a native polypeptide are included, provided the biological activity of the protein is maintained. A modified protein may be biologically functionally equivalent to its native counterpart.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, binding sites to substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 1 shows the codons that encode particular amino acids. A proteinaceous molecule has “homology” or is considered “homologous” to a second proteinaceous molecule if one of the following “homology criteria” is met: 1) at least 30% of the proteinaceous molecule has sequence identity at the same positions with the second proteinaceous molecule; 2) there is some sequence identity at the same positions with the second proteinaceous molecule and at the nonidentical residues, at least 30% of them are conservative differences, as described herein, with respect to the second proteinaceous molecule; or 3) at least 30% of the proteinaceous molecule has sequence identity with the second proteinaceous molecule, but with possible gaps of nonidentical residues between identical residues. As used herein, the term “homologous” may equally apply to a region of a proteinaceous molecule, instead of the entire molecule. If the term “homology” or “homologous” is qualified by a number, for example, “50% homology” or “50% homologous,” then the homology criteria, with respect to 1), 2), and 3), is adjusted from “at least 30%” to “at least 50%.” Thus it is contemplated that there may homology of at least 30%, 35%, 40%, 45%, 50%, 55%, 60%, 65%, 70%, 75%, 80%, 85%, 90%, 95%, or more between two proteinaceous molecules or portions of proteinaceous molecules.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of modified polypeptides according to the invention is the use of peptide mimetics. Mimetics are peptide-containing molecules that mimic elements of protein secondary structure. See, e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation modified protein molecules having many of the natural properties of a native protein, but with altered and, in some cases, even improved characteristics.

a. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope or other tag, to facilitate targeting or purification of the fusion protein. The use of 6×His and GST (glutathione S transferase) as tags is well known. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

Immunotoxins are specifically contemplated as an embodiment of the present invention. An immunotoxin is a cytotoxic compound comprising at least a portion of an antibody and a portion of a toxin molecule. The antibody and the toxin may be fused or conjugated to each other. More detail about immunotoxins is provided infra.

b. Conjugated Proteins

The present invention further provides conjugated polypeptides, such as translated proteins, polypeptides and peptides that are linked to at least one agent to form an conjugate. Of particular use are antibody conjugates, in which the antibody portion targets the agent to a particular site. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety that may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins.”

i. Linkers/Coupling Agents

Multiple peptides or polypeptides may be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin. Alternatively, peptides or polypeptides may be joined to an adjuvant. Amino acids such as selectively-cleavable linkers, synthetic linkers, or other amino acid sequences may be used to separate proteinaceous moieties.

4. Protein Purification

While some of the embodiments of the invention involve recombinant proteins, the invention concerns also methods and processes for purifying proteins, including endogenous polypeptides and peptides and recombinant polypeptides and peptides. Generally, these techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC. In addition, the conditions under which such techniques are executed may be affect characteristics, such as functional activity, of the purified molecules.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur. A “substantially purified” protein or peptide

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, about 96%, about 97%, about 98%, about 99%, about 99.2%, about 99.4%, about 99.6%, about 99.8%, about 99.9% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

The use of a peptide tag in combination with the methods and compositions of the invention is also contemplated. A tag takes advantage of an interaction between two polypeptides. A portion of one of the polypeptides that is involved in the interaction may used as a tag. For instance, the binding region of glutathione S transferase (GST) may be used as a tag such that glutathione beads can be used to enrich for a compound containing the GST tag. An epitope tag, which an amino acid region recognized by an antibody or T cell receptor, may be used. The tag may be encoded by a nucleic acid segment that is operatively linked to a nucleic acid segment encoding a modified protein such that a fusion protein is encoded by the nucleic acid molecule. Other suitable fusion proteins are those with β-galactosidase, ubiquitin, hexahistidine (6×His), or the like.

5. Antibodies

In certain embodiments, the present invention involves antibodies. For example, some aspects of the invention involve identifying a patient in need of treatment or evaluating the type of spinal cord injury a patient has. These aspects may involve antibodies, for example to assay IL-1β levels. As used herein, the term “antibody” is intended to refer broadly to any immunologic binding agent such as IgG, IgM, IgA, IgD and IgE. The techniques for preparing and using various antibody-based constructs and fragments are well known in the art. Means for preparing and characterizing antibodies are also well known in the art (See, e.g., Harlow and Lane, “Antibodies: A Laboratory Manual,” Cold Spring Harbor Laboratory, 1988; incorporated herein by reference).

6. Immunodetection Methods

As discussed, in some embodiments, the present invention concerns immunodetection methods for binding, purifying, removing, quantifying and/or otherwise detecting biological components such as antigenic regions on polypeptides and peptides. The immunodetection methods of the present invention can be used to identify antigenic regions of a peptide, polypeptide, or protein that has therapeutic implications, particularly in reducing the immunogenicity or antigenicity of the peptide, polypeptide, or protein in a target subject.

Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle and Ben-Zeev, 1999; Gulbis et al, 1993; De Jager et al., 1993; and Nakamura et al., 1987, each incorporated herein by reference.

III. Nucleic Acid Compositions

A. Polynucleotides Encoding Native Proteins or Modified Proteins

The present invention concerns polynucleotides, isolatable from cells, that are free from total genomic DNA and that are capable of expressing all or part of a protein, polypeptide, or peptide, such as IL-1ra. Alternatively, the present invention may concern an antisense or ribozyme that inhibits the expression of a particular protein. The polynucleotide may encode a native protein that may be manipulated to encode a modified protein. Alternatively, the polynucleotide may encode a modified protein, or it may encode a polynucleotide that will be used to make a fusion protein with a modified protein. Recombinant proteins can be purified from expressing cells to yield active proteins. Thus, embodiments of the invention include the use of nucleic acids encoding all or part of an IL-1ra, such as those identified by GenBank accession numbers (nucleic acid and amino acid sequences). Such nucleic acids include all or part of the nucleic acids disclosed herein and may further include other nucleic acid sequences, such as those that allow IL-1ra to be expressed in a heterologous cell, to be targeted to a particular cell type or site, to be more stable; or to add an agent that also confers a therapeutic benefit. Thus, it is contemplated that any of the methods and compositions discussed herein with respect to nucleic acids may be applied with respect to manipulating nucleic acids encoding all or part of an IL-1ra.

As used herein, the term “DNA segment” refers to a DNA molecule that has been isolated free of total genomic DNA of a particular species. Therefore, a DNA segment encoding a polypeptide refers to a DNA segment that contains wild-type, polymorphic, or mutant polypeptide-coding sequences yet is isolated away from, or purified free from, total mammalian or human genomic DNA. Included within the term “DNA segment” are a polypeptide or polypeptides, DNA segments smaller than a polypeptide, and recombinant vectors, including, for example, plasmids, cosmids, phage, viruses, and the like.

As used in this application, the term “polynucleotide” refers to a nucleic acid molecule that has been isolated free of total genomic nucleic acid. Therefore, a “polynucleotide encoding a native polypeptide” refers to a DNA segment that contains wild-type or polymorphic polypeptide-coding sequences isolated away from, or purified free from, total mammalian or human genomic DNA. Therefore, for example, when the present application refers to the function or activity of IL-1ra, which is encoded by a IL-1ra-encoding polynucleotide, it is meant that the polynucleotide encodes a molecule that has the IL-1 antagonistic activity of IL-1ra.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a particular polypeptide from a given species may be represented by natural variants that have slightly different nucleic acid sequences but, nonetheless, encode the same protein (see Table 1 above).

Similarly, a polynucleotide comprising an isolated or purified wild-type, polymorphic, or mutant polypeptide gene refers to a DNA segment including wild-type, polymorphic, or mutant polypeptide coding sequences and, in certain aspects, regulatory sequences, isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. A nucleic acid encoding all or part of a native or modified polypeptide may contain a contiguous nucleic acid sequence encoding all or a portion of such a polypeptide of the following lengths: about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs. Particularly contemplated are such contiguous nucleic acids of IL-1ra-encoding polynucleotides, such as those identified herein by Genbank accession number.

In particular embodiments, the invention concerns isolated DNA segments and recombinant vectors incorporating DNA sequences that encode a wild-type, polymorphic, or modified polypeptide or peptide that includes within its amino acid sequence a contiguous amino acid sequence in accordance with, or essentially corresponding to a native polypeptide. IL-1ra polypeptides and peptides are specifically contemplated. The term “recombinant” may be used in conjunction with a polypeptide or the name of a specific polypeptide, and this generally refers to a polypeptide produced from a nucleic acid molecule that has been manipulated in vitro or that is the replicated product of such a molecule.

In other embodiments, the invention concerns isolated DNA segments and recombinant vectors incorporating DNA sequences that encode a polypeptide or peptide that includes within its amino acid sequence a contiguous amino acid sequence in accordance with, or essentially corresponding to the polypeptide.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode full-length polypeptide from any source or encode a truncated version of the polypeptide, for example a truncated IL-1ra polypeptide, such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targetting or efficacy. As discussed above, a tag or other heterologous polypeptide may be added to the modified polypeptide-encoding sequence, wherein “heterologous” refers to a polypeptide that is not the same as the modified polypeptide.

In a non-limiting example, one or more nucleic acid constructs may be prepared that include a contiguous stretch of nucleotides identical to or complementary to the a particular nucleic acid, such as the IL-1ra-encoding nucleic acid. A nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

The DNA segments used in the present invention encompass biologically functional equivalent modified polypeptides and peptides, for example, a modified IL-1ra. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein, to reduce toxicity effects of the protein in vivo to a subject given the protein, or to increase the efficacy of any treatment involving the protein.

1. Vectors

Native and modified polypeptides may be encoded by a nucleic acid molecule comprised in a vector. Also comprised in a vector may be a nucleic acid sequence encoding an antisense or ribozyme molecule. Such a vector may be used to deliver a therapeutic polypeptide or peptide, such as IL-1ra for spinal cord injuries, to cells or tissue. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference. In addition to encoding a modified polypeptide such as modified IL-1ra, a vector may encode non-modified polypeptide sequences such as a tag or targetting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targetting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. Control sequences include promoters and enhancers, initiation signals and internal ribosome binding sites, termination signals, splice sites, as well as sequences that can be used for manipulation of nucleic acids, such as multiple cloning sites and restriction enzyme sites generally. In addition to control sequences that govern transcription and translation, vectors and expression vectors may contain nucleic acid sequences that serve other functions as well and are well known to those of skill in the art.

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al, 1998), D1A dopamine receptor gene (Lee, et al., 1997), insulin-like growth factor II (Wu et al., 1997), human platelet endothelial cell adhesion molecule-1 (Almendro et al., 1996).

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses. Appropriate yeast cells include Saccharomyces cerevisiae, Saccharomyces pombe, and Pichia pastoris.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

2. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

3. Antisense and Ribozymes

Other compounds may be used in conjunction with IL-1ra, including inhibitors of Par4, or any other pro-apoptotic protein. Such inhibitors include antisense and ribozyme molecules that target Par4 to achieve a reduction or elimination of Par4 protein in a cell. Thus, it is contemplated that nucleic acid molecules that are identical or complementary to all or part of a Par-4 encoding sequence are included as part of the invention.

a. Antisense Molecules

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction may be used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

b. Ribozymes

The use of Par4-specific ribozymes is an embodiment of the present invention. The following information is provided in order to compliment the earlier section and to assist those of skill in the art in this endeavor.

Ribozymes are RNA-protein complexes that cleave nucleic acids in a site-specific fashion. Ribozymes have specific catalytic domains that possess endonuclease activity (Kim and Cech, 1987; Gerlack et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990; Sioud et al., 1992). Recently, it was reported that ribozymes elicited genetic changes in some cell lines to which they were applied; the altered genes included the oncogenes H-ras, c-fos and genes of HIV. Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Several different ribozyme motifs have been described with RNA cleavage activity (reviewed in Symons, 1992). Examples that would be expected to function equivalently for the down regulation of Par4 include sequences from the Group I self splicing introns including tobacco ringspot virus (Prody et al., 1986), avocado sunblotch viroid (Palukaitis et al., 1979; Symons, 1981), and Lucerne transient streak virus (Forster and Symons, 1987). Sequences from these and related viruses are referred to as hammerhead ribozymes based on a predicted folded secondary structure.

Other suitable ribozymes include sequences from RNase P with RNA cleavage activity (Yuan et al., 1992; Yuan and Altman, 1994), hairpin ribozyme structures (Berzal-Herranz et al., 1992; Chowrira et al., 1993) and hepatitis δ virus based ribozymes (Perrotta and Been, 1992). The general design and optimization of ribozyme directed RNA cleavage activity has been discussed in detail (Haseloff and Gerlach, 1988; Symons, 1992; Chowrira, et al., 1994; and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in Par4-targeted ribozymes is simply a matter of preparing and testing a given sequence, and is a routinely practiced “screening” method known to those of skill in the art.

4. Method of Nucleic Acid Transfer

Suitable methods for nucleic acid delivery to effect expression of compositions of the present invention are believed to include virtually any method by which a nucleic acid (e.g., DNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

5. Viral Vectors

A retroviral construct carrying IL-1ra has already been constructed, called MFG-IRAP (Baragi, 2000). It is contemplated that IL-1ra may be delivered to glial and neurons at the site of a spinal cord injury by a number of ways including through the use of viral vectors. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

B. Nucleic Acid Detection

In addition to their use in directing the expression of desirable proteins, polypeptides and/or peptides, the nucleic acid sequences disclosed herein have a variety of other uses. Detection of nucleic acids are encompassed by the invention, for example, for identifying a subject in need of treatment for spinal cord injury or evaluating the spinal cord injury. Hybridization, amplification, and other techniques involving detection of nucleic acids may be implemented in practicing the invention.

IV. Combination Therapies

Therapies for spinal cord injuries, known to one of skill in the art, may be used in combination with the therapeutic agents, IL-1 receptor antagonists, of the present invention. Thus, in order to increase the effectiveness of the therapy using a therapeutic agent, or expression construct coding therefor, it may be desirable to combine these compositions with other therapeutic agents effective in the treatment of spinal cord injury such as, but not limited to, those described below. For example, one can use one can use the IL-1 receptor antagonist-based therapy in conjunction with methylprednisolone and/or one or more neurotrophic factors (mentioned earlier), and/or other therapeutic methods and compositions. As used herein, “therapeutic agents” refers to agents that have a therapeutic effect with respect to methods of the invention, particularly methods of treating spinal cord injury.

The other therapy may precede or follow the IL-1 receptor antagonist-based therapy by intervals ranging from minutes to days to weeks. In embodiments where the other macular or retinal degeneration therapy and the therapeutic agent-based therapy are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the other neurodegeneration therapy and the IL-1 receptor antagonist-based therapy will be required to effect therapy for spinal cord injury. Various combinations may be employed, where the other neurodegeneration therapy is “A” and the IL-1 receptor antagonist-based therapy treatment is “B,” as exemplified below:

A/B/A B/A/B B/B/A A/A/B B/A/A A/B/B B/B/B/A B/B/A/
B A/A/B/B A/B/A/B A/B/B/A B/B/A/A B/A/B/A B/A/A/B
B/B/B/A A/A/A/B B/A/A/A A/B/A/A A/A/B/A A/B/B/B B/
A/B/B B/B/A/B

Other combinations also are contemplated. The exact dosages and regimens can be suitable altered by those of ordinary skill in the art.

V. Formulations and Routes of Administration

Pharmaceutical compositions of the present invention comprise an effective amount of one or more therapeutic agents dissolved or dispersed in a pharmaceutically acceptable carrier. It is an aspect of the invention that an IL-1 receptor antagonist be prepared for pharmaceutical administration. Additional therapeutic agents include methylprednisolone and neurotrophic factors.

A. Formulations

The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one IL-1 receptor antagonist or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The therapeutic agents may comprise different types of carriers depending on whether it is to be administered in solid, liquid or aerosol form, and whether it need to be sterile for such routes of administration as injection. The present invention can be administered intraocularly, intravenously, intradermally, intraarterially, intraperitoneally, intracranially, topically, intramuscularly, intraperitoneally, subcutaneously, intravesicularlly, mucosally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Therapeutic agents of the invention may be dissolved or dispersed in artificial cerebrospinal fluid (ACSF) for administration to the spinal cord.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 nanogram/kg/body weight, about 5 nanogram/kg/body weight, about 10 nanogram/kg/body weight, about 50 nanogram/kg/body weight, about 100 nanogram/kg/body weight, about 200 nanogram/kg/body weight, about 350 nanogram/kg/body weight, about 500 nanogram/kg/body weight, 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The IL-1 receptor inhibitor, such as IL-1ra may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof. The carrier can also be a controlled release matrix that allows a slow release of the active ingredients mixed or admixed therein. Examples of such controlled release matrix material include, but are not limited to, sustained release biodegradable formulations described in U.S. Pat. No. 4,849,141 to Fujioka et al., U.S. Pat. No. 4,774,091 to Yamashira, U.S. Pat. No. 4,703,108 to Silver et al., and Brem et al., 1991, all of which are incorporated herein by reference.

In other embodiments, one may use drops, solutions or sprays, aerosols or inhalants in the present invention. Such compositions are generally designed to be compatible with the target tissue type.

In certain embodiments the therapeutic agent is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

B. Routes of Administration

Those of skill in the art are well aware of how to apply gene delivery to in vivo and ex vivo situations. For viral vectors, one generally will prepare a viral vector stock. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, or 1×10¹² infectious viral particles to the patient. Similar figures may be extrapolated for liposomal or other non-viral formulations by comparing relative uptake efficiencies. Formulation as a pharmaceutically acceptable composition is discussed below.

The phrases “pharmaceutically acceptable” or “pharmacologically acceptable” refer to molecular entities and compositions that do not produce an adverse, allergic, or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in the therapeutic compositions is contemplated. Supplementary active ingredients, such as other therapeutic agents, can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including cremes, lotions, mouthwashes, inhalants and the like.

The active compounds of the present invention can be formulated for parenteral administration, e.g., formulated for injection via the intravenous, intramuscular, intrathoracic, subcutaneous, or even intraperitoneal routes. The preparation of an aqueous composition that contains a compound or compounds that increase the bioavailibility of IL-1ra or a neurotrophic factor will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of therapeutic compositions according to the present invention will be via any common route so long as the target tissue is available via that route. In cases where the present invention is used as a viral vector, a primary consideration will be the desired location for the heterologous sequences carried by the vector. Routes of administration include oral, nasal, buccal, rectal, vaginal or topical. Administration will be by orthotopic, intradermal subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery to the lung is contemplated. Volume of the aerosol is between about 0.01 ml and 0.5 ml. Similarly, a preferred method for treatment of colon-associated disease would be via enema. Volume of the enema is between about 1 ml and 100 ml. Direct intratumoral injection is the preferred mode, with continuous intratumoral perfusion a more specific embodiment.

In certain embodiments, it may be desirable to provide a continuous supply of therapeutic compositions to the patient. For intravenous or intraarterial routes, this is accomplished by drip system. For topical applications, repeated application would be employed. For various approaches, delayed release formulations could be used that provided limited but constant amounts of the therapeutic agent over and extended period of time. For internal application, continuous perfusion, for example with a viral vector carrying a heterologous nucleic acid segment, of the region of interest may be preferred. This could be accomplished by catheterization, post-operatively in some cases, followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the injections are administered. It is believed that higher doses may be achieved via perfusion, however.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, Remington's Pharmaceutical Sciences, 1990). Some variation in dosage will necessarily occur depending on the condition of the subject being treated. The person responsible for administration will, in any event, determine the appropriate dose for the individual subject.

Furthermore, it may be desirable to administer a pharmaceutical composition of the invention containing a neuronal survival factor locally to the area in need of treatment, this may be achieved by, for example, local infusion during surgery, by injection, by means of a catheter, or by means of an implant, wherein such implant can be of a porous, non-porous, or gelatinous material, including membranes, such as silastic membranes or fibers.

An effective amount of the therapeutic composition is determined based on the intended goal. In this case, the intended goal is to effect a therapeutic effect on the spinal cord. A therapeutic effect includes, but is not limited to, reduction in apoptosis of neuronal and/or glial cells of or around the spinal cord; reduction or delay of neurodegeneration in or around spinal cord, recovery of motor function after injury to spinal cord; decrease in pain at or near spinal cord; reduction in long-term damage to neuronal and/or glial cells of or around spinal cord, extension of neuronal or glial cell existence, and improvement in behavioral reflexes after injury to spinal cord. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability, and toxicity of the particular therapeutic substance.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

As used herein, the term in vitro administration refers to manipulations performed on cells removed from an animal, including, but not limited to, cells in culture. The term ex vivo administration refers to cells that have been manipulated in vitro, and are subsequently administered to a living animal. The term in vivo administration includes all manipulations performed on cells within an animal.

In certain aspects of the present invention, the compositions may be administered either in vitro, ex vivo, or in vivo. In certain in vitro embodiments, an expression construct encoding a modified protein may be transduced into a host cell. The transduced cells can then be used for in vitro analysis, or alternatively for in vivo administration.

U.S. Pat. Nos. 4,690,915 and 5,199,942, both incorporated herein by reference, disclose methods for ex vivo manipulation of blood mononuclear cells and bone marrow cells for use in therapeutic applications.

In vivo administration of the compositions of the present invention are also contemplated. Examples include, but are not limited to, catheterization, by infusion of appropriate transducing compositions through the portal vein via a catheter (Bao, 1996), use of a pump in combination with a catheter. Additional examples include direct injection of tumors with the instant transducing compositions, and either intranasal or intratracheal (Dong, 1996) instillation of transducing compositions to effect transduction of lung cells.

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally and/or using aerosol, injection, infusion, continuous infusion, localized perfusion bathing target cells directly or via a catheter and/or lavage.

Administration can be either rapid as by injection or over a period of time as by slow infusion or administration of slow release formulation. For treating tissues in the central nervous system, administration can be by injection or infusion into the cerebrospinal fluid (CSF). Administration can be with one or more agents capable of promoting penetration of therapeutic agents across the blood-brain barrier.

Therapeutic agents can also be linked or conjugated with agents that provide desirable pharmaceutical or pharmacodynamic properties. For example, IL-1ra can be coupled to any substance known in the art to promote penetration or transport across the blood-brain barrier such as an antibody to the transferrin receptor, and administered by intravenous injection. (See for example, Friden et al., 1993, which is specifically incorporated by reference). Furthermore, IL-1ra can be stably linked to a polymer such as polyethylene glycol to obtain desirable properties of solubility, stability, half-life and other pharmaceutically advantageous properties. (See for example Davis et al., 1978; Burnham, 1994, which are specifically incorporated by reference). Preferably, a therapeutic agent is administered with a carrier such as liposomes or polymers containing a targeting moiety to limit delivery of the therapeutic agent to targeted cells. Examples of targeting moieties include but are not limited to antibodies, ligands or receptors to specific cell surface molecules.

For nonparenteral administration, the compositions can also include absorption enhancers which increase the pore size of the mucosal membrane. Such absorption enhancers include sodium deoxycholate, sodium glycocholate, dimethyl-.beta.-cyclodextrin, lauroyl-1-lysophosphatidylcholine and other substances having structural similarities to the phospholipid domains of the mucosal membrane.

The therapeutic agents can also be administered in a cell based delivery system in which a DNA sequence encoding an IL-1 receptor antagonist is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635, which are all specifically incorporated by reference.

B. Lipid Compositions

In certain embodiments, the present invention concerns a novel composition comprising one or more lipids associated with a polynucleotide or polypeptide of the claimed invention. A lipid is a substance that is characteristically insoluble in water and extractable with an organic solvent. Compounds than those specifically described herein are understood by one of skill in the art as lipids, and are encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A person of ordinary skill in the art would know how to produce and use lipid compositions for use with the present invention.

1. Liposome/Nucleic Acid Combinations

It is contemplated that when the liposome composition comprises a cell or tissue specific nucleic acid, this technique may have applicability in the present invention. In certain embodiments, lipid-based non-viral formulations provide an alternative to viral gene therapies. Although many cell culture studies have documented lipid-based non-viral gene transfer, systemic gene delivery via lipid-based formulations has been limited. A major limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Solodin et al., 1995; Liu et al., 1995; Thierry et al., 1995; Tsukamoto et al., 1995; Aksentijevich et al., 1996).

An exemplary method for targeting viral particles to cells that lack a single cell-specific marker has been described (U.S. Pat. No. 5,849,718).

The addition of targeting ligands for gene delivery for the treatment of conditions or injury permits the delivery of genes whose gene products are more toxic than do non-targeted systems. Examples of the more toxic genes that can be delivered includes pro-apoptotic genes such as Bax and Bak plus genes derived from viruses and other pathogens such as the adenoviral E4orf4 and the E.coli purine nucleoside phosphorylase, a so-called “suicide gene” which converts the prodrug 6-methylpurine deoxyriboside to toxic purine 6-methylpurine. Other examples of suicide genes used with prodrug therapy are the E. coli cytosine deaminase gene and the HSV thymidine kinase gene.

It is also possible to utilize untargeted or targeted lipid complexes to generate recombinant or modified viruses in vivo. For example, two or more plasmids could be used to introduce retroviral sequences plus a therapeutic gene into a hyperproliferative cell. Retroviral proteins provided in trans from one of the plasmids would permit packaging of the second, therapeutic gene-carrying plasmid. Transduced cells, therefore, would become a site for production of non-replicative retroviruses carrying the therapeutic gene. These retroviruses would then be capable of infecting nearby cells. The promoter for the therapeutic gene may or may not be inducible or tissue specific.

Similarly, the transferred nucleic acid may represent the DNA for a replication competent or conditionally replicating viral genome, such as an adenoviral genome that lacks all or part of the adenoviral E1a or E2b region or that has one or more tissue-specific or inducible promoters driving transcription from the E1a and/or E1b regions. This replicating or conditional replicating nucleic acid may or may not contain an additional therapeutic gene.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Materials and Methodology Employed in Examples 1-5

Animal Model for Spinal Cord Injury

Contusion injury to the rat spinal cord is closely related to the contusion/cyst type of injury (Basso et al., 1996) which is most often observed in humans (Bunge et al., 1993; van de Meent et al., 1996), so we used that injury model in these experiments. Sprague Dawley male rats (200-300 g) were anesthetized, their backs shaved, the area washed with the antiseptic betadine and a laminectomy performed over spinal segment T8. The spinal cord was injured by dropping an impactor probe (10 g, 2 mm diameter) from a distance of 12.5 mm onto the cord at spinal segment T8. Following injury, the animals were maintained in the anesthetized state and sacrificed without revival from anesthesia if sampling was to occur during the next several hours. For longer-term experiments, the wound was closed by suturing the muscle and fascia and the skin closed with surgical staples.

Three groups of rats (sham, and two groups of injured rats) were given either artificial cerebrospinal fluid (ACSF; sham and one group of injured rats) or rmIL-1 receptor antagonist (recombinant mouse IL-1ra, R&D Systems; 750 ng/ml). All solutions were administered using an ALZET minipump (Alzet, 1003D, 1 μl/h) implanted in the rats subcutaneously. A catheter (polyethylene tubing, PE-60) was connected to the minipump and directed to the site of injury.

Statistical Analysis

All results are expressed as means±SE. Statistical analyses for each parameter compared the values obtained with the vehicle (ACSF) infusion in the injured and uninjured (sham) spinal cord with those obtained with infusion of a test solution in the injured spinal cord. The two-sample, unpaired t-test was applied when the data passed the normality and equal variance test. Whenever normality was not achieved, all comparisons were tested with the non-parametric Mann-Whitney U-test as well. Differences were considered to be significant only at P-values less then 0.05.

Ribonuclease Protection Assay for Cytokine Gene Expression

The portion of the spinal cord containing segments T5-T11 was removed from anesthetized animals to provide tissue for the analyses. The animal was perfused transcardially with 100-150 ml of 0.9% saline; segments were then marked based on the location of dorsal roots and the desired section of the cord removed. The isolated length of cord was immediately placed in dry ice and allowed to freeze fully. The piece was then cut into seven sections, each constituting one spinal cord segment. Total RNA was prepared from frozen spinal cord segments (T5-T11) using TRI-Reagent (Molecular Research Center). Spinal segments were homogenized in 200 μl of TRI-Reagent, and total RNA extracted in chloroform, ethanol precipitated, and stored at −80° C. Cytokine expression was determined by a Ribonuclease Protection Assay (RPA). The Riboquant Multi-Probe RNAse Protection Assay kit (PharmaMingen) was used for the measurement of 11 rat cytokine mRNAs. Templates were used to generate a ³²P-labeled anti-sense RNA probe set that was hybridized in excess to target RNAs. Total RNA (10 μg) was hybridized with the labeled probe sets followed by RNAase treatment and analysis of protected bands on a denaturing 5% PAGE. Gels were subjected to PhosphorImage analysis and bands were quantified by densitometry. Relative cytokine mRNA levels were calculated by normalizing specific cytokine species measured to the ribosomal RNA L32 mRNA, which was included among probes provided as an internal control.

ELISA IL-1β Detection Assay

ELISA was used to measure IL-1β protein levels in cytosolic fractions of different spinal cord segments. Cytoplasmic fractions were isolated according to methods modified from Schreiber et al. (1989). Spinal cord segments were isolated and homogenized in ice cold buffer containing HEPES 10 mM, MgCl₂ 10 mM, EDTA 0.1 mM, EGTA 0.1 mM, DTT 1 mM, 2 μg/ml pepstatin, aprotinin, leupeptin and 0.5 mM PMSF. The homogenates were centrifuged (15 min, 8000 rpm) and cytosolic fractions extracted. The assay was performed according to the BioSource Int. protocol for solid phase sandwich ELISA using antibodies specific for rat IL-1β. The sample absorbance was read with an ELISA plate reader (Dynex) adjusted to 450 nm, and the concentration was determined based on a standard IL-1β curve established with recombinant rat Il-1β.

ELISA Cell Death Detection Assay

The apoptosis ELISA was performed according to the Roche Cell Death Detection ELISA protocol (Roche, Inc.). The assay is based on sandwich-enzyme-immunoassay principle using mouse monoclonal antibodies directed against DNA and histones, respectively. This allows the specific determination of mono- and oligonucleosomes in the cytoplasmic fraction of spinal cord tissue. Cytoplasmic fraction was isolated according to the protocol described above. The sample absorbance was read with an ELISA plate reader (Dynex) at 405 nm.

Caspase-3 Activity Colorimetric Assay

Spinal cord segments were isolated and homogenized in ice cold buffer according to the protocol described above. The homogenates were centrifuged (15 min, 8000 rpm) and the cytosolic fraction extracted. Homogenates (50 μg of protein) were incubated overnight in a buffer supplemented with a synthetic peptide (DEVD), which is a specific substrate for Caspase-3. DEVD was conjugated with a chromogen p-nitroanilide (p-NA) (Colorimetric Caspase-3 Assay, Biosource Int.). Free p-NA light absorbance was quantified using a microplate reader at 405 nm.

### Example 1

### IL-1β mRNA and Protein Levels Increase Rapidly After Contusion to the Spinal Cord

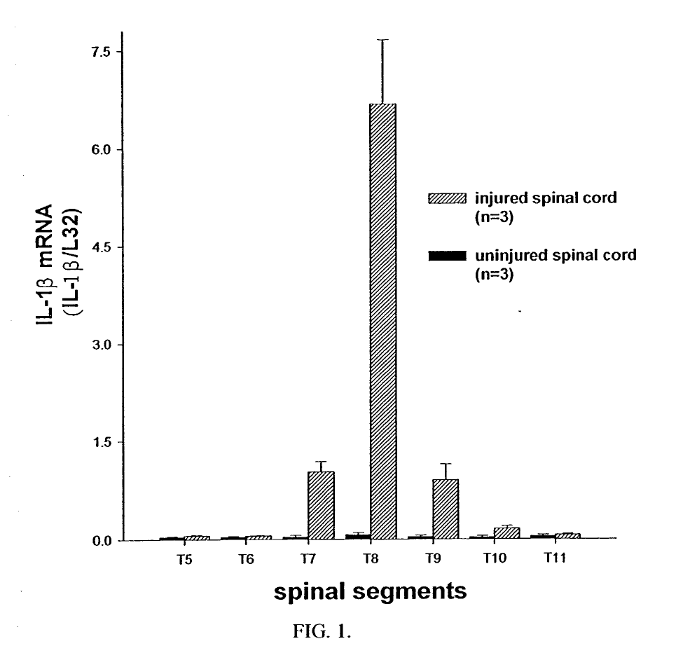

Of the cytokines tested, increases in IL-1β and TNFα transcription occur most rapidly after SCI, with the IL-1β mRNA increase being most robust. As shown in FIG. 2, IL-1β mRNA levels peaked at 1 h, decreased by 6 h and returned to sham levels 72 h after injury (not shown). The observed increases were localized to the injury site T8 and were not present in the more rostral T5 or the more caudal T11 segments (FIG. 1). Therefore, all further measurements of apoptosis were restricted to the T8 injury site and compared to T5 spinal segments in order to use each animal as its own control. FIG. 2 shows IL-1β protein levels at 6 and 72 h after injury. Six hours after injury there is a significant increase in IL-1β protein levels at the site of injury (p=0.03). Three days after injury, protein levels decrease significantly, even compared to the control levels.

### Example 2

### Apoptosis and Caspase-3 Activity Increase in Parallel After SCI

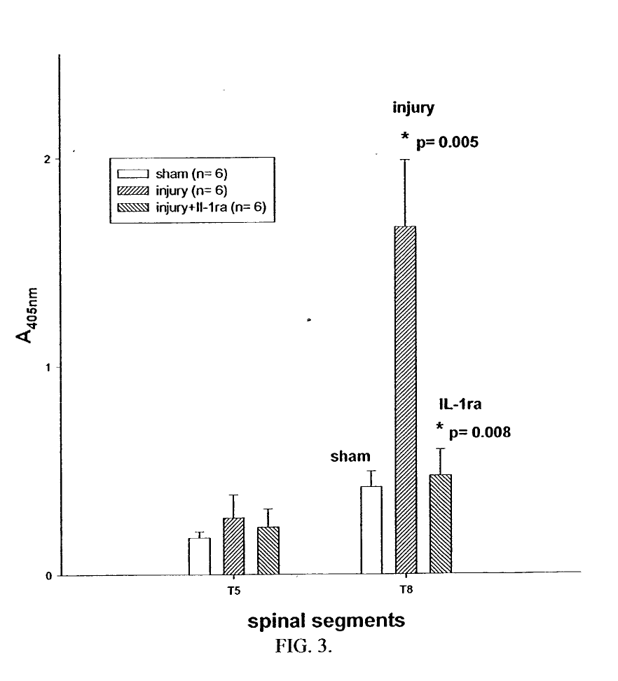

As shown in FIG. 3, there was a very significant increase (4×) (p=0.05) in free nucleosome levels at the site of injury, compared to levels of free nucleosomes in sham-treated spinal cords. This observation shows that there is a robust increase in apoptosis at T8, at 72 h after injury.

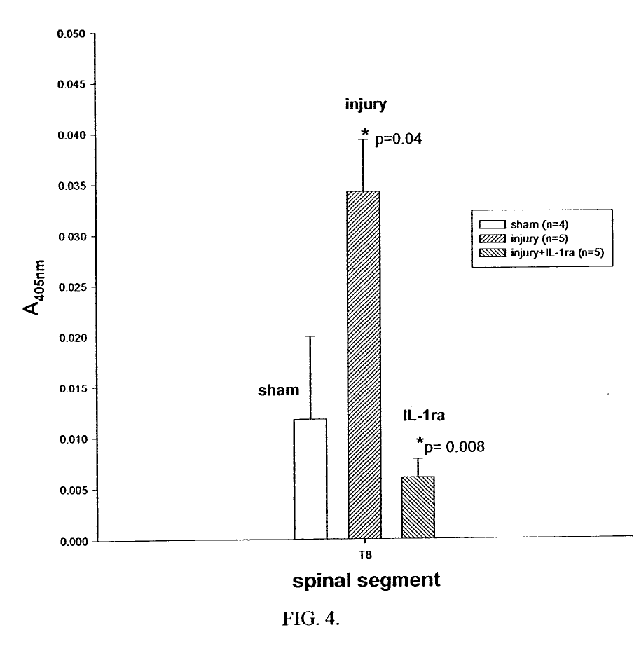

To confirm this finding, caspase-3 activity in spinal cord cytosolic fractions was measured with a colorimetric assay (FIG. 4). Caspase-3 activity is a hallmark of apoptosis in the injured CNS (Ni et al., 1998; Springer et al., 1999). FIG. 4 shows the levels of caspase-3 activity in sham-treated and injured spinal cord at the injury site, 72 h after injury. Caspase-3 activity increased significantly after injury (p=0.04), similar (approximately 4×) to the increase in apoptosis (FIG. 3). Apoptosis increased 4-fold by 72 hours after injury. Also, at segment T5, which is away from the site of injury at T8, there was no significant increase in apoptosis by day 3, indicating that during the first 3 days after injury, apoptosis probably contributes to cell death at the site of injury. Moreover, the spatial and temporal profiles of apoptosis after SCI correlate well with the spatiotemporal distribution of IL-1β expression (FIG. 1).

Administration of IL-1ra decreased caspase-3 activity (FIG. 4), with a pattern similar to that observed for apoptosis in these animals (FIG. 3). The administration of IL-1ra significantly reduced cell death after SCI (FIG. 3), resulting in no significant differences in the levels of cell death observed in sham-treated vs. injured spinal cords injected with IL-1ra.

### Example 3

Apoptosis and caspase-3 activity decrease after SCI in the presence of the IL-1 receptor antagonist (IL-1ra) IL-1ra is a competitive inhibitor of both forms of IL-1 (IL-1α and IL-1β; Arend et al., 1998). IL-1ra from a variety of sources generally share the same structure and have a high level of amino acid sequence homology. In particular, the rat and mouse IL-1ra exhibit the same general structure and have a high amino acid sequence homology (Arend et al., 1998). Commercially available mouse recombinant IL-1ra (rmIL-1ra) was used in these experiments. The rmIl-1ra was administered for 72 h directly into the injury site via catheters connected to ALZET minipumps. The concentration of rmIL-1ra used (750 ng/ml) was selected based on published reports (Plata-Salaman et al., 1995; Lundkvist et al., 1999). Considering that the pumping rate of ALZET osmotic minipumps was 1 μl/h, the rats received about 70 μl of a solution, or approximately 500 ng of rmIL-1ra over the time of drug administration.

As shown in FIG. 3, the chronic administration of rmIL-1ra during the first 72 h after injury significantly reduced SCI-induced apoptosis (p=0.08). There was no significant difference between the levels of apoptosis in the sham-treated spinal cords and the injured spinal cords treated with rmIL-1ra.

Similar effects were observed when caspase-3 activity was measured in cytosolic fractions of injured spinal cord treated with rmIL-1ra. (FIG. 4). There was a significant (p=0.008) decrease in caspase-3 activity at the injury site of the rats treated with rmIL-1ra for 72 h.

### Example 4

### Recovery of Locomotor Activity Following Treatment with IL-1ra

One indication of the success of the disclosed method is the recovery of locomotor activity in injured rats treated with rmIL-1ra. Locomotor recovery was measured in an open field test and scored on the BBB scale. The BBB scale is a 21-point scale for testing normal overground locomotion. See Basso et al., 1995. It is based on the observation of hindlimb movements of a rat freely moving in an open field. In this scale, parameters such as joint movements, the ability for weight support, limb coordination, foot placement and gait stability are rated. A score of 0 indicates no observable hindlimb movement, a score of 21 points reflects normal locomotion.

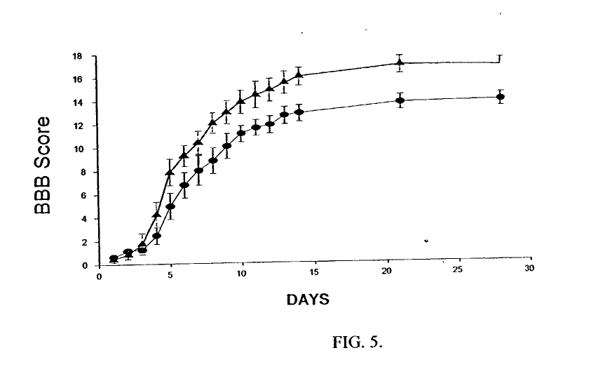

When scored over a period of little as 5 days post treatment, there was a significant improvement in locomotor activity in the subjects treated. This improvement increased over time, resulting in a stable (at 30 days) advantage of treated subjects over sham controls (FIG. 5). Administration of IL-1ra directly at the site of injury prevents cell death, particularly apoptosis, and allows for better motor recovery after SCI.

### Example 5

### Treatment of SCI Via Adenoviral Vector Delivery of Polynucleotides Encoding IL-1ra

Delivery of IL-1ra to the injury site may be substantially improved, both in efficacy and in relative cost by the use of gene-therapy methods. Adenoviral vectors are one avenue of delivering biologically active IL-1ra proteins.

Adenoviral vectors containing nucleic acid sequences encoding the relevant IL-1ra protein, e.g. human IL-1ra, are constructed and provided to the injury site or injured subject, where the expression of the IL-1ra sequence results in effective treatment. See, for example, Yang et al. (1997) Br. Res. 751 (2), pp. 181, testing the overexpression of human IL-1ra in mice.

Briefly, recombinant adenovirus will be produced carrying the nucleic acid encoding an IL-1ra linked to an internal CMV-IE or other, suitable promoter (may be tissue-specific), and followed by SV40 polyadenylation (pA) signal. Methods for production of recombinant adenovirus are well known in the art. See, for example, International Patent Application No. PCT/US00/19392, incorporated herein by reference.

Three groups of rats (sham, and two groups of injured rats as described above) are injected at the site of injury with either artificial cerebrospinal fluid (ACSF; sham and one group of injured rats) or recombinant adenovirus containing the nucleic acid sequence of human IL-1 receptor antagonist (recombinant human IL-1ra). Apoptosis, and caspase-III activity is measured as described at 1, 6, 24, and 72 hours post injury and treatment. Expression of human IL-1ra at the site of injury will result in significantly lowered levels of apoptosis and caspase-III activity. Injured and treated and sham control groups will be scored for locomotor recovery on the BBB scale as above. Expression of IL-1ra provided by adenoviral vector significantly promotes improvement in locomotor recovery.

### Example 6

### Transcriptional Changes Underlying IL-1ra Effect

The inhibition of IL-1 receptors to prevent cell death in injured SC and improve motor recovery was further investigated. Signal transduction requires binding of IL-1 receptor, IL-1RI (80 kDa), to the membrane-bound IL-1 receptor accessory protein (IL-1RAcP). IL-1 receptor activates several ubiquitous transcription factors (NF-κB, AP-1, AFP) and some key intracellular signaling molecules (JNK, IP3 or PKC-kinases; Auron, 1998), suggesting that the inhibition of IL-1 receptors will likely affect many intracellular pathways. To answer this question transcriptional changes were evaluated 24 h after SCI in injured vs. injured spinal cord treated with IL-1ra. Affymetrix DNA microarrays (RGA, 8799 probes) were used, 5 samples per experimental group.

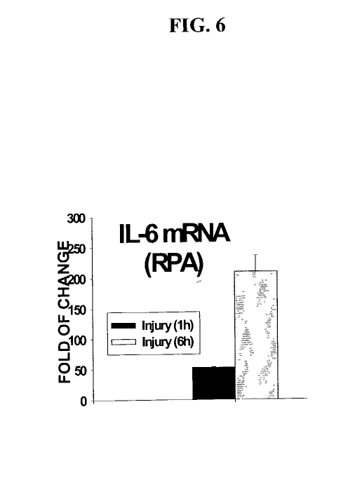

Analysis of DNA microarray data showed that about 15% of SCI-induced changes in mRNAs (24 h after SCI) were reversed in the presence of IL-1ra. To validate IL-1ra intervention, the effect of IL-1ra treatment was evaluated on mRNA levels of inflammatory genes known to be directly regulated by IL-1β. SCI-induced increases were observed in IL-6, IFNγ, IFγ receptor, TGFβ, Activin receptor, Fibronectin, Integrin and iNOS mRNAs, and then observed to be reversed in the presence of IL-1ra. IL-6, TGFβ, Integrin and iNOS were also affected by MK-801. For example, in the first six hours after SCI, IL-6 mRNA increased 210-fold (FIG. 6). It appears IL-1ra prevents over-production of TGFβ, iNOS and Integrin—all molecules with the role in cell death and secondary damage after SCI.

In addition to iNOS, significantly increased mRNA levels of COX-2 were found to be reduced in the presence of IL-1ra, which is consistent with previous findings that IL-1 increases iNOS and COX-2 mRNA levels in neurons in vitro (Serou et al., 1999; Igwe et al., 2001).

Furthermore, the increased presence of the Hypoxia-Inducible Factor (HIF)-activated mRNAs coding for glycolytic enzymes, p21/Waf1 and Cyclins (Semenza, 2000), were detected after SCI. HIF is a protein with protective antioxidant activity, whose transcription is induced in the presence of Reactive Oxygen Species (ROS). All these SCI-induced increases in mRNA levels were reversed in the presence of rIL-1ra.

Furthermore, it was found that IL-1ra increases SCI-induced reduction in Calpastatin, which is Calpain inhibitor. Therefore, DNA microarray analyses of IL-1ra treated injured spinal cord suggest that IL-1, robustly upregulated early after SCI is capable of activating Caspase-3 via different pro-apoptotic cellular pathways, predominantly via excessive production of ROS.

DNA microarray analysis of IL-1ra treated injured spinal cords revealed possible SCI-induced death pathways not affected by IL-1ra. For example, SCI decreases mRNA expression of the following neuroprotective molecules: Neurotrophin 3 (NT3;), CRF receptors (Elliott-Hunt et al., 2002) and Endothelin receptor (Ehrenreich et al., 2000).

Neurotrophin-3 increases survival of axotomised neurons (Novikova et al., 2000) and enhances axonal regeneration after SCI (Coumans et al., 2001).

Additionally, SCI stimulated up-regulation of pro-apoptotic Prostate apoptosis response-4 (Par-4).

### Example 7

### Combined Effect of IL-1ra, NT3, and Par4 Antisense

The current data suggest that early up-regulation of Par-4 plays a pivotal role in ischemic neuronal death in animal models of stroke and cardiac arrest. Par-4 levels increase in hippocampal and striatal neurons in rats after transient forebrain ischemia; Par-4 levels increased within 6 hours of reperfusion and remained elevated in neurons undergoing apoptosis 3 days later. A Par-4 antisense oligonucleotide protected cultured hippocampal neurons against apoptosis induced by chemical hypoxia and significantly reduced focal ischemic damage in mice (Culmsee et al., 2001). It has been shown that fluid percussion brain injury also increases Par-4 (Dhillon et al., 2001).

Therefore, a cocktail that includes IL-1ra and either 1) Par4 antisense and/or 2) NT3 (polypeptide or encoding nucleic acid) will be administered to injured rats to evaluate any effect on cell loss and locomotor recovery.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the references identified in the text, to the extent that they provide exemplary procedural or other details supplementary to those set forth herein, are specifically incorporated herein by reference.

U.S. Pat. No. 4,554,101

U.S. Pat. No. 4,683,202

U.S. Pat. No. 4,684,611

U.S. Pat. No. 4,690,915

U.S. Pat. No. 4,703,108

U.S. Pat. No. 4,774,091

U.S. Pat. No. 4,849,141

U.S. Pat. No. 4,879,236

U.S. Pat. No. 4,952,500

U.S. Pat. No. 5,028,592

U.S. Pat. No. 5,199,942

U.S. Pat. No. 5,302,523

U.S. Pat. No. 5,322,783

U.S. Pat. No. 5,354,855

U.S. Pat. No. 5,384,253

U.S. Pat. No. 5,464,765

U.S. Pat. No. 5,538,877

U.S. Pat. No. 5,538,880

U.S. Pat. No. 5,550,050

U.S. Pat. No. 5,550,318

U.S. Pat. No. 5,563,055

U.S. Pat. No. 5,563,055

U.S. Pat. No. 5,580,859

U.S. Pat. No. 5,589,466

U.S. Pat. No. 5,591,616

U.S. Pat. No. 5,610,042

U.S. Pat. No. 5,656,610

U.S. Pat. No. 5,702,932

U.S. Pat. No. 5,736,524

U.S. Pat. No. 5,780,448

U.S. Pat. No. 5,789,215

U.S. Pat. No. 5,849,718

U.S. Pat. No. 5,871,986

U.S. Pat. No. 5,928,906

U.S. Pat. No. 5,945,100

U.S. Pat. No. 5,981,274

U.S. Pat. No. 5,994,624

U.S. Pat. No. 6,221,676

U.S. Pat. No. 6,274,624

U.S. Pat. No. 6,280,732

U.S. Pat. No. 6,284,540

U.S. Patent Publication 20010011126

Patent Appl. No. PCT/US00/19392

PCT Application WO 92/19195

PCT Application WO 94/09699

PCT Application WO 94/25503

PCT Application WO 95/01203

PCT Application WO 95/05452

PCT Application WO 95/06128

PCT Application WO 96/02286

PCT Application WO 96/02646

PCT Application WO 96/40871

PCT Application WO 96/40959

PCT Application WO 97/12635

Aksentijevich et al., Hum. Gene Ther., 7(9):1111-1122, 1996.

Almendro et al., J. Immunol., 157(12):5411-5421, 1996.

Arend et al., Ann. Rev. Immunol. 16:27-55, 1998.

Ausubel et al., In: Current Protocols in Molecular Biology, John, Wiley & Sons, Inc, NY, 1994.

Baichwal and Sugden, In: Gene Transfer, Kucherlapati (ed.), New York, Plenum Press, 117-148, 1986.

Bao et al., Hum. Gene Ther., 7(3):355-65, 1996.

Baragi, Curr. Opin. Mol Ther., 2(2):216-20, 2000.

Barany and Merrifield, In: The Peptides, Gross and Meienhofer (eds.), Academic Press, New York, 1-284, 1979.

Bartholdi et al., Brain Res., 762(1-2), 173 (1997)

Bartholdi et al., Eur. J. Neurosci., 9(7):1422-38, 1997.

Basso et al., Exp. Neurol., 139:244, 1996.

Basso et al., J. Neurotrauma, 12:1-21, 1995.

Berzal-Herranz et al., Genes Dev., 6(1):129-134, 1992.

Brem et al., J. Neurosurg., 74: 441-446, 1991.

Bunge et al., In Advances in Neurology. Seil (Ed.), Raven Press, NY, 59:75-89 (1993).

Burnham, Am. J. Hosp. Pharm., 51:210-218, 1994.

Capaldi et al., Biochem. Biophys. Res. Comm., 74(2):425-433, 1977.

Cech et al., Cell, 27(3 Pt 2):487-496, 1981.

Chen and Okayama, Mol. Cell Biol., 7(8):2745-2752, 1987.

Chowrira et al., Biochemistry, 32(4):1088-1095, 1993.

Clark et al., J. Neurotrauma, 5:499-506, 1994.

Coumans et al., J. Neurosci., 21(23):9334-9344, 2001.

Coupar et al., Gene, 68:1-10, 1988.

Crowe et al., Nature Med., 3:73-76, 1997.

Culmsee et al., J. Cereb. Blood Flow Metab., 21(4):334-343, 2001.

Davis et al., Enzyme Eng., 4:169-73, 1978.

De Jager et al., Semin. Nucl. Med., 23(2):165-79, 1993.

Dinarello, Ann. N.Y. Acad. Sci., 856:1-11, 1998(a).

Dinarello, Intl. Rev. Immunol., 16:457, 1998(b).

Dinarello, Methods, 19(1):121-32, 1999.

Dong, Hum. Gene. Ther., 7(3):319-331, 1996.

Doolittle et al., Methods Mol. Biol., 109:215-37, 1999.

Dusart et al., Eur. J. Neurosci., 6:712, 1994.

Ehrenreich et al., Neuroscience, 95(4):993-1001, 2000.

Elliott-Hunt et al., J. Neurochem., 80(3):416-425, 2002.

Emery et al., J. Neurosurg., 89:911, 1998.

Fechheimer et al., Proc. Natl. Acad. Sci. USA, 84:8463-8467, 1987.

Felgner et al., Proc. Natl. Acad. Sci. USA, 84(21):7413-7417, 1987.

Fenton et al., Int. J. Immunopharmacol., 14(3):401, 1992.

Forster and Symons, Cell, 49(2):211-220, 1987.

Fraley et al., Proc. Natl. Acad. Sci. USA, 76:3348-3352, 1979.

Franco et al., Free Radic. Biol. Med., 27(9-10):1122-1132, 1999.

Friden et al., Science, 259:373-377, 1993.

Friedlander et al., J. Experimental Medicine, 184:717, 1996.

Friedlander et al., J. Experimental Medicine, 185:933, 1997.

Friedmann, Science, 244:1275-1281, 1989.

Gendelman et al., J. Leukoc. Biol., 56:389, 1994.

Gerlach et al., Nature (London), 328:802-805, 1987.

Gopal, Mol. Cell Biol., 5:1188-1190, 1985.

Graham and van der Eb, Virology, 52:456-467, 1973.

Griffin et al., Proc. Natl. Acad. Sci. USA., 86:7611, 1989.

Gulbis et al., Hum. Pathol., 24(12):1271-85, 1993.

Harland and Weintraub, J. Cell Biol., 101:1094-1099, 1985.

Harlow et al., “Antibodies: A Laboratory Manual” (Cold Spring Harbor Laboratory, 1988).

Haseloff and Gerlach, Nature, 334:585-591, 1988.

Hayashi et al., J. Neurotrama., 17(3):203, 2000.

Hermonat and Muzycska, Proc. Natl. Acad. Sci. USA, 81:6466-6470, 1984.

Hetier et al., J. Neurosci. Res., 21:391, 1988.

Holmin et al., J. Neurosurgery, 92(1):108, 2000.

Horwich et al., Virol., 64:642-650, 1990.

Hulsebosch et al., J. Neurotrauma, 17:1205, 2000.

Inouye and Inouye, Nucleic Acids Res., 13: 3101-3109, 1985.

Irmler et al., J. Exp. Med. 181(5):1917, 1995.

Johnson et al., In: Biotechnology And Pharmacy, Pezzuto et al., eds., Chapman and Hall, New York, 1993.

Joyce, Nature, 338:217-244, 1989.

Kaeppler et al., Plant Cell Reports, 9:415-418, 1990.

Kaneda et al., Science, 243:375-378, 1989.

Kato et al, J. Biol. Chem., 266:3361-3364, 1991.

Katoh et al., Neurosci. Lett., 216:9, 1996.

Kim and Cech, Proc. Natl. Acad. Sci. USA, 84:8788-8792, 1987.

Klusman et al., Brain Research, 762:173, 1997.

Kraus et al. FEBS Lett., 428(3):165-170, 1998.

Kyte and Doolittle, J. Mol. Biol., 157(1):105-32, 1982.

Lareyre et al., J. Biol. Chem., 274(12):8282-8290, 1999.

Lawrence et al., Eur. J. Neurosci., 10: 1188, 1998.

Lebedeva et al., Biochem. Biophys. Acta., 1353(1):32, 1997.

Lee et al., J. Auton. Nerv. Syst., 74(2-3):86-90, 1997.

Lee et al., Chin. J. Physiol., 42(1):17-23, 1999.

Li et al., Cell Biol. 11:261, 2000.

Lieber and Strauss, Mol. Cell. Biol., 15: 540-551, 1995.

Liu et al., Biochim Biophys Acta, 1240(2):277-84, 1995.

Liu et al., J. Neurosci., 17:5395, 1997.

Loddick et al., J. Cere. Blood Flow Metab., 16:932, 1996.

Lundkvist et al., Am. J. Physiol., 276:r644, 1999.

Martin et al., Brain Res. Bulletin, 46:281, 1998.

Martin et al., Exp. Neurol., 130:362, 1994.

McGeer and Rogers, Neurology, 42(2):447-449, 1992.

Merrifield, Science, 232: 341-347, 1986.

Michel and Westhof, J. Mol. Biol., 216:585-610, 1990.

Nakahara et al., J. Neuropathol. Exp. Neurol., 58(5):442, 1999.

Nakamura et al., In: Handbook of Experimental Immunology (4^{th} Ed.), Weir et al., (eds). 27(1), Blackwell Scientific Publ., Oxford, 1987.

Nakao et al., Mol. Cell Biochem., 209(1-2):113-118, 2000.

Ni Eidhin et al., Mol. Microbiol., 30:245-257, 1998.

Ni et al., J. Cereb. Blood Flow Metab., 18:248, 1998.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau et al., Methods Enzymol., 149:157-176, 1987.

Nomoto et al., Gene, 236(2):259-271, 1999.

Novikova et al., Eur. J. Neurosci., 12(2):776-80, 2000.

Omirulleh et al., Plant Mol. Biol., 21(3):415-428, 1993.

Palukaitis et al., Virology, 99:145-151, 1979.

Perriman et al., Gene, 113:157-163, 1992.

Perrotta and Been, Biochemistry, 31(1):16-21, 1992

Plata-Salaman et al., Am. J. Physiol., 269:r1032, 1995.

Potrykus et al., Mol. Gen. Genet., 199(2):169-177, 1985.

Prody et al., Science, 231:1577-1580, 1986.

Rabuffetti et al., J. Neurosci., 20(12):4398, 2000.

Raivich et al., Brain Res. Rev., 30:77, 1999.

Reinhold-Hurek and Shub, Nature, 357:173-176, 1992.

Relton et al., Exp. Neurol., 138:206, 1996.

Remington's Pharmaceutical Sciences, 18th Ed. Mack Printing Company, pp. 1289-1329, 1990.

Ridgeway, In: Vectors: A survey of molecular cloning vectors and their uses. Rodriguez and Denhardt, eds. Stoneham: Butterworth, pp. 467-492, 1988.

Rippe et al., Mol. Cell Biol., 10:689-695, 1990.

Rothwell et al., Int. Rev. Neurobiol., 40:281, 1997.

Rothwell, J. Physiol., 514:3-17, 1999.

Saito et al., J. Neurotrauma, 17(2):173, 2000.

Sambrook, et al., Molecular Cloning, A Laboratory Manual, 2nd ed., Cold Spring Harbor Laboratory, Cold Spring, Harbor, N.Y., 1989.

Sanderson et al., J. Cereb. Blood Flow Metab., 19:1118, 1999.

Sarver et al., Science, 247:1222-1225, 1990.

Scanlon et al., Proc. Natl. Acad. Sci. USA, 88:10591-10595, 1991.

Schreiber et al., Nucleic Acid Res., 17(15):6419

Semenza, J. Appl. Physiol., 88(4): 1474-80, 2000.

Short et al., Spinal Cord, 38:273, 2000.

Sioud et al., J. Mol. Biol., 223:831-835, 1992.

Solodin et al., Biochemistry, 34(41):13537-44, 1995.

Springer et al., Nature Neurosci., 5(8):943, 1999.

Stewart and Young, In: Solid Phase Peptide Synthesis, 2d. ed., Pierce Chemical Co., 1984.

Streit et al., Exp. Neurology, 152:74, 1998.

Symons, Annu. Rev. Biochem., 61:641-671, 1992.

Symons, Nucl. Acids Res., 9:6527-6537, 1981.

Tam, et al., J. Am. Chem. Soc., 105:6442, 1983.

Taoka and Okajima, Prog. Neurobiol., 56(3):341-358, 1998.

Tasker et al., In: Textbook of Pain, Wall (Ed.), Churchill Livingston, N.Y., 2:154-180, 1989.

Temin, In: Gene Transfer, Kucherlapati (ed.), NY, Plenum Press, 149-188, 1986.

Thierry et al., Proc. Natl. Acad. Sci. USA, 92(21):9742-6, 1995.

Thompson et al., Nature Genet., 9:444-450, 1995.

Tonai et al., J. Neurochem, 72(1), 302 (1999)

Tsukamoto et al., Nat. Genet., 9(3):243-248, 1995.

Tsumaki et al., J. Biol. Chem., 273(36):22861-22864, 1998.

Van de Meent et al., J. Neurotrauma, 12:741, 1996.

Vezzani et al., J. Neurosci., 19(12):5054, 1999.

Wada et al., J. Neurosurg, (Spine 1), 91:98-104, 1999.

Wang et al., Brain Res., 759(2):190, 1997.

Wong et al., Gene, 10:87-94, 1980.

Wu and Wu, J. Biol. Chem., 262:4429-4432, 1987.

Xie et al. Biophys. J., 67(3):1247-1251, 1994.

Yang et al., Br. Res. 751(2):181, 1997.

Yong et al., J. Neurotrauma, 15:459-472, 1998.

Yuan and Altman, Science, 263:1269-1273, 1994.

Yuan et al., Proc. Natl. Acad. Sci. USA, 89:8006-8010, 1992.

Zhao-Emonet et al., Biochim Biophys Acta, 1442(2-3):109-119, 1998.

Zhu et al., Chin J Biotechnol., 9(4):257-61, 1993.

What is claimed is:

1. A method of treating spinal cord injury in which the spinal cord is not completely severed in a subject comprising administering a therapeutically effective amount of IL-1 receptor antagonist (IL-1ra) to the site of injury.

2. The method of claim 1, wherein the subject is a human.

3. The method of claim 1, wherein the subject is a horse, a dog, or a cow.

4. The method of claim 1, further comprising identifying a subject in need of such treatment.

5. The method of claim 1, wherein the IL-1 receptor antagonist is human IL-1ra.

6. The method of claim 1, wherein the IL-1 receptor antagonist is mouse IL-1ra.

7. The method of claim 1, wherein the IL-1 receptor antagonist is recombinant IL-1ra.

8. The method of claim 7, wherein the IL-1 receptor antagonist is recombinant mouse IL-1ra.

9. The method of claim 7, wherein the IL-1 receptor antagonist is recombinant human IL-1ra.

10. The method of claim 1, wherein the IL-1ra is administered by introduction of a nucleic acid encoding the IL-1ra.

11. The method of claim 10, wherein said nucleic acid sequence is provided in a vector.

12. The method of claim 11, wherein the vector is a viral vector.

13. The method of claim 12, wherein the viral vector is an adenovirus, retrovirus, vaccinia adeno-associated virus vector.

14. The method of claim 1, further comprising identifying the type of spinal cord injury.

15. The method of claim 4, wherein the spinal cord injury is traction on the spinal cord.

16. The method of claim 4, wherein the spinal cord injury is contusion.

17. The method of claim 4, wherein the spinal cord injury is partial transection.

18. The method of claim 1, further comprising administration of the IL-1 receptor antagonist over a therapeutically effective time period.

19. The method of claim 18, wherein the time period is within one hour of the time of the spinal cord injury to 72 hours after the spinal cord injury.

20. The method of claim 18, wherein the time period begins within about 1 hour of injury to 72 hours after the spinal cord injury.

21. The method of claim 18, wherein the time period is between 24 hours and 72 hours in length.

22. The method of claim 18, wherein the time period is between 3 and 6 days in length.

23. The method of claim 18, wherein the administration is chronic.

24. The method of claim 1, wherein the administration is repeated at least once.

25. The method of claim 1, wherein the amount of the IL-1 receptor antagonist administered is between 1 and 1000 nanograms per kilogram body weight per hour.

26. The method of claim 1, wherein the amount of the IL-1 receptor antagonist administered is between 1 and 100 nanograms per kilogram body weight per hour.

27. The method of claim 1, wherein the amount of the IL-1 receptor antagonist administered is between 1 and 10 nanograms per kilogram body weight per hour.

28. The method of claim 1, wherein the IL-1 receptor antagonist is dispersed or dissolved in a pharmaceutically acceptable carrier.

29. The method of claim 1, wherein the IL-1 receptor antagonist is administered to the site of injury via a catheter.

30. The method of claim 29, wherein the antagonist is administered by pump.

31. The method of claim 1, further comprising administering methylpredisolone.

32. The method of claim 31, wherein the methylpredisolone is administered before the administration of the IL-1 receptor antagonist.

33. The method of claim 31, wherein the IL-1 receptor antagonist is administered before the administration of the methylpredisolone.

34. The method of claim 31, wherein the IL-1 receptor antagonist is administered with the methylpredisolone.

35. The method of claim 1, further comprising administering a neurotrophic factor.

36. The method of claim 35, wherein the neurotrophic factor is bFGF, aFGF, CNTF, NGF, BDNF, GDNF, NT3, NT4/5, IGF-1, IGF-II, NT-4, IL-1β, TNFα., TGF-β, TGF-β1, NTN, persephin (PSP), artemin, or AL-1.

37. The method of claim 35, wherein the neurotrophic factor is NT3.

38. The method of claim 35, further comprising administering a Par4 antisense molecule.

39. The method of claim 38, wherein the IL-1 receptor antagonist, NT3, Par4 antisense molecule are administered at the same time.

40. A method of treating a spinal cord injury comprising:
a) identifying a subject in need of such treatment,
b) identifying the type of spinal cord injury; and
c) administering a therapeutically effective amount of IL-1 receptor antagonist to the site of injury over a therapeutically effective time period.

41. A pharmaceutical composition for the treatment of spinal cord injury at the site of injury comprising IL-1 receptor antagonist and methylpredisolone.

42. A pharmaceutical composition for the treatment of spinal cord injury at the site of injury comprising IL-1 receptor antagonist and a neurotrophic factor.

43. The composition of claim 42, wherein the neurotrophic factor is bFGF, aFGF, CNTF, NGF, BDNF, GDNF, NT3, NT4/5, IGF-1, IGF-II, NT-4, IL-1β, TNFα., TGF-β, TGF-β1, NTN, persephin (PSP), artemin, or AL-1.

44. The composition of claim 43, wherein the neurotrophic factor is NT3.

45. The composition of claim 44, further comprising a Par4 antisense molecule.

46. A method of treating spinal cord injury in which the spinal cord is not completely severed in a subject comprising administering a therapeutically effective amount of recombinant human IL-1 receptor antagonist (IL-1ra) to the site of injury.
